(19) United States
(12) Patent Application Publication
(10) Pub. No.: US 2009/0143841 A1
(43) Pub. Date: Jun. 4, 2009

(54) Potential Application Apparatus

(75) Inventor: Takuji Hara, Saitama (JP)

Correspondence Address: COOK ALEX LTD, SUITE 2850, 200 WEST ADAMS STREET, CHICAGO, IL, 60606, US

(73) Assignees: Bestec Corporation, Saitama (JP); Takuji Hara and Akikuni Hara, Saitama (JP)

(21) Appl. No.: 11/910,550

(22) PCT Filed: Apr. 5, 2006
(86) PCT No.: PCT/JP2006/307224

§ 371 Date: Oct. 3, 2007

(30) Foreign Application Priority Data
Apr. 5, 2005 (JP) 2005 109243

(51) Int. Cl. A61N 1/00

(52) U.S. Cl. 607/76

(57) ABSTRACT

Provided is a potential application apparatus capable of preventing hypertrophy of adipose cells, thereby securely preventing deposition of fat and controlling obesity effectively without any side effects.

The potential application apparatus has an influence on adipose cells by applying, at a predetermined frequency, a superposed waveform potential consisted of a negative direct current potential plus an alternating current potential to a human body while insulating it.

### TECHNICAL FIELD

The present invention relates to a potential application apparatus for having an influence on adipose cells which will be a cause of obesity, thereby preventing deposition of them.

### BACKGROUND ART

Various methods such as medication, administration of supplements such as ion supply drink, magnet therapy and ultrasonic therapy are employed for preventing obesity. In Patent Document 1, as illustrated in FIG. 1, there is described a belt for removing or reducing subcutaneous fat which has accumulated too much in a human body and at the same time, preventing the deposition of fat. The belt has an electrode obtained by weaving a copper fibrous material and another electrode made from an aluminum material arranged at a predetermined distance at appropriate positions on one side of a belt body. It is used by winding it around the human body while bringing these two electrodes into contact with the skin to allow the skin to serve as a conducting circuit. A weak current flowing between these electrodes is utilized for achieving the objects. This document, however, does not include specific clinical data showing that the belt can prevent deposition of fat so that its effectiveness on the obesity has not yet been confirmed.

[Patent Document 1] Japanese Patent Laid-Open No. 2000-37464

### DISCLOSURE OF THE INVENTION

### Problems to be Solved by the Invention

With the drawbacks of the conventional apparatuses in view, the present invention has been made. The present invention is a first invention that shows the concrete data of animal experiments by which the effectiveness of a potential therapy apparatus on the deposition of fat is confirmed. An object of the present invention is to provide a potential application apparatus capable of preventing adipose cell hypertrophy, securely preventing deposition of fat, and suppressing obesity effectively without any side effects.

### Means for Solving the Problems

A potential application apparatus according to the invention applies a superposed waveform potential, which has been consisted of a negative direct current potential plus an alternating current potential, to a human body at a predetermined frequency, while insulating the human body.

Another potential application apparatus according to the invention applies a superposed waveform potential, which has been consisted of a negative direct current potential plus an alternating current potential of from 50 to 1500 V, to a human body at a frequency of from 5 to 100 kHz, preferably from 30 to 40 kHz while insulating the human body.

A further potential application apparatus according to the invention applies a superposed waveform potential, which has been consisted of a negative direct current potential plus an alternating current potential of from 50 to 1500 V and controlling a ratio of the superposed waveform potential on the positive side to the potential on the negative side to 1:2 or less, to a human body at a frequency of from 5 to 100 kHz, preferably from 30 to 40 kHz, while insulating the human body.

A still further potential application apparatus according to the invention is equipped with a first electrode for applying a superposed waveform potential, which has been consisted of a negative direct current potential plus an alternating current potential of from 50 to 1500 V, at a frequency of from 5 to 100 kHz, preferably from 30 to 40 kHz, and a second electrode on the side opposite to the first electrode, which serves as a ground electrode.

A still further potential application apparatus according to the invention comprises a first electrode for applying an alternating current potential of from 50 to 1500 V at a frequency of from 5 to 100 kHz, preferably from 30 to 40 kHz and a second electrode on the side opposite to the first electrode for applying a negative direct current potential; and forms a superposed waveform potential.

A still further potential application apparatus according to the invention has an influence on adipose cells by applying a composite waveform potential obtained by adding a fluctuation waveform potential to a superposed waveform potential.

A still further potential application apparatus according to the invention is used by attaching the electrode for applying a superposed waveform potential to an adipose cell deposition inhibited site.

A still further potential application apparatus according to the invention is applied by winding a band roll to which electrodes have been attached around an adipose cell deposition inhibited site.

The potential application apparatus according to the present invention makes it possible to prevent adipose cell hypertrophy, securely prevent deposition of fat, and suppress obesity effectively without any side effects.

### BRIEF DESCRIPTION OF THE DRAWINGS

FIG. 1 is a schematic view illustrating a conventional potential application apparatus.

FIG. 2 is a schematic view illustrating a potential application apparatus obtained by applying the principle of a potential therapy apparatus to a cage of mice.

FIG. 3a shows a waveform composed only of an alternating current potential.

FIG. 3b shows a superposed waveform consisted of a negative direct current potential plus an alternating current potential.

FIG. 3c includes superposed waveforms consisted of a negative direct current potential plus an alternating current potential and different in a ratio of a potential on the positive side and that on the negative side.

FIG. 4a is a schematic view illustrating a chair-type potential application apparatus.

FIG. 4b is a schematic view illustrating a person sitting on the chair while placing his or her feet on a conducting electrode plate and having a ground electrode attached to the lumbar.

FIG. 5 is a cross-sectional view illustrating a conducting electrode structure obtained by covering a plate made of metals, carbon or the like with an insulator.

FIG. 6 is a cross-sectional view illustrating a ground electrode structure obtained by covering a conductor plate covered with an insulator.

FIG. 7a is a schematic view of a mat-type potential application apparatus.

FIG. 7b is a cross-sectional view of the potential application apparatus of FIG. 7a.

FIG. 8a is a schematic view of a dome-type potential application apparatus.

FIG. 8b is a schematic view illustrating the apparatus placed at the leg portion of a person who is lying.

FIG. 8c is an exploded view of the apparatus.

FIG. 9a is a schematic view of a hollow-cylinder-type potential application apparatus.

FIG. 9b is a schematic view illustrating the apparatus 9a attached to a brachial portion.

FIG. 9c is a schematic view illustrating the apparatus 9a attached to a leg portion.

FIG. 10 is a schematic view of a roll-type potential application apparatus.

FIG. 11a illustrates a circuit for forming a superposed waveform potential to be applied to a drive electrode.

FIG. 11b illustrates a circuit for forming a superposed waveform potential by applying an alternating current potential to a first electrode and a negative direct current potential to a second electrode.

FIG. 12 is Table 1 including data showing the effect brought about by the application of a superposed waveform potential.

FIG. 13 is a graph of Table 1 in FIG. 12.

FIG. 14 is Table 2 including data showing the results of histopathological test.

FIG. 15 is Table 3 including data showing that potential application by a potential application apparatus did not cause any change in organs and tissues.

### DESCRIPTION OF REFERENCE NUMBERS

10: Potential application apparatus
20: Chair
32: Insulation mat
12: First electrode (ground electrode)
14: Second electrode (drive electrode)
34, 52, 60, 68, 74: Conducting electrode plate
36, 54, 64, 70, 76: Ground electrode
42, 46: Plate made of metals, carbon or the like
44, 48: Insulator
50: Mat
58: Roof member
72: Roll member

### BEST MODE FOR CARRYING OUT THE INVENTION

The best mode for carrying out the invention will next be described specifically referring to accompanying drawings.

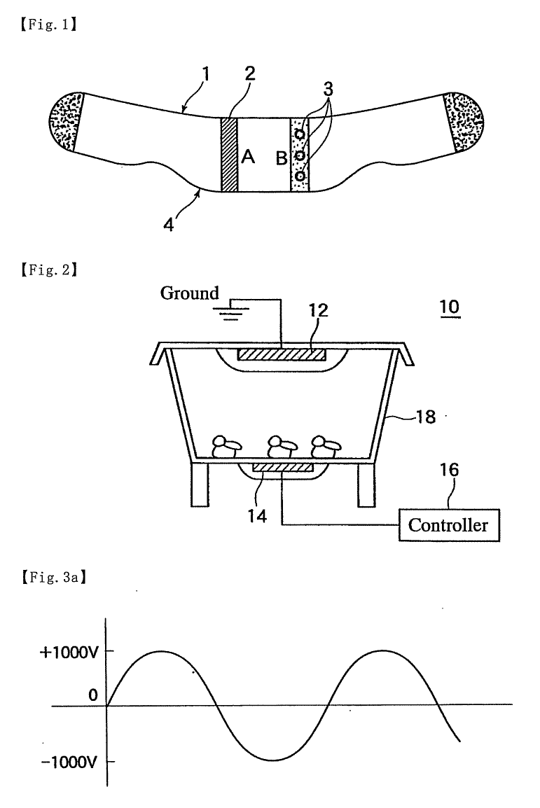

FIG. 2 is a schematic view illustrating a potential application apparatus for applying a potential to a cage containing obese mice. This apparatus will hereinafter be described. The potential application apparatus is equipped with a first electrode and a second electrode which are arranged vertically and opposite to each other; and a controller for controlling the voltage and frequency of the first electrode and second electrode. In the potential application apparatus 10 in this drawing, the first electrode 12 is used as a ground electrode, while the second electrode is used as a drive electrode. The ground electrode 12 and drive electrode 14 are attached to the inside of a lid of the cage containing mice and outside of a bottom plate of the cage, respectively. The cage is made of plastic.

To the drive electrode 14, a superposed waveform potential composed of an alternating current potential of from 50 to 1500 V and a negative direct current potential of from 100 to 500 V, preferably from 200 to 400 V is applied. The current is from 1 to 1.2 mmA and the frequency is from 5 to 100 kHz, preferably from 30 to 40 kHz.

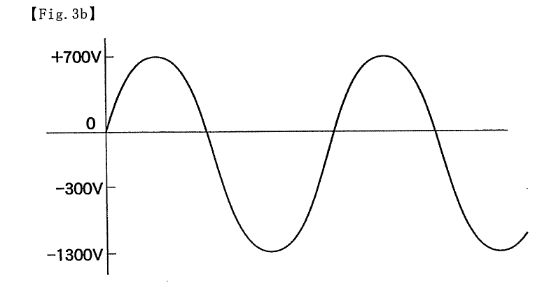

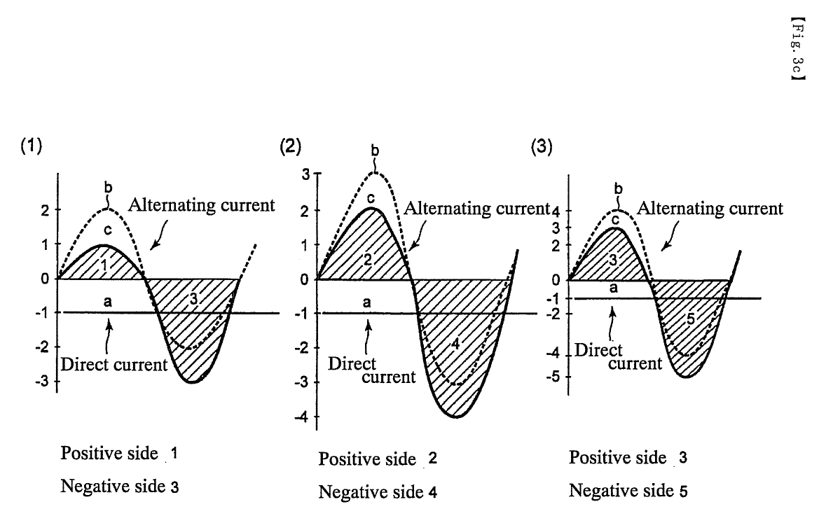

FIG. 3(a) illustrates a waveform composed only of an alternating current potential, while FIG. 3(b) illustrates a superposed waveform obtained by adding a negative direct current potential to an alternating current potential. The superposed waveform may be added with a fluctuation waveform.

Thus, the potential application apparatus is composed of the first electrode 12 and the second electrode 14 arranged vertically opposite to each other, and a controller 16 for controlling the voltage and frequency to be applied to these electrodes to predetermined values. It is equipped with the first electrode and the second electrode disposed on the side opposite thereto for applying a superposed waveform potential, which has been consisted of a negative direct current potential plus an alternating current potential of from 50 to 1500 V, at frequency of from 5 to 100 kHz, preferably from 30 to 40 kHz.

FIG. 3(c) illustrates a superposed waveform consisted of a negative direct current potential plus an alternating current potential. In this drawing, a represents a direct current potential to be applied, b represents an alternating current potential to be applied, and c represents a superposed waveform composed of the direct current potential a and the alternating current potential b. In the drawing, (1) is the superposed waveform (c) in which a ratio of a potential on the positive side to that on the negative side is 1:3, (2) is the superposed waveform (c) in which a ratio of a potential on the positive side to that on the negative side is 1:2, and (3) is the superposed waveform (c) in which a ratio of a potential on the positive side to that on the negative side is 3:5.

In this Example, a superposed waveform in which a ratio of a potential on the positive side to that on the negative side is 1:2 is employed. A ratio of a potential on the positive side to that on the negative side in the superposed waveform may be 1:2 or less.

The potential application apparatus is equipped with a first electrode for applying, at a frequency of from 5 to 100 kHz, preferably from 30 to 40 kHz, an alternating current potential of from 50 to 1500 V and a second electrode disposed on the side opposite to the first electrode and used for applying a negative direct current potential.

In a cage of this potential application apparatus, db/db mice which were said to have a characteristic of human diabetic nephropathy were housed and an influence of potential application by a potential therapy apparatus on diabetic nephropathy was studied. The db/db mice are said to naturally develop marked diabetic symptoms such as obesity, overeating and hyperinsulinemia and thus have a characteristic of human diabetic nephropathy.

### <Test System>

### Animals Used for the Test

Kind of animals: mouse

Strain: C57BL/KsJ-db/dbJc1

Sex: Male

The number of mice purchased: 30 (obtained: 30, used: 27)

Age in week: Eight weeks old when they are purchased, Nine weeks old when their use is started.

Weight range: from 35.1 to 40.5 g when they are purchased, from 40.2 to 4.57 when they are classified into groups.

Supply source: CLEA Japan, Inc

Reason for selecting this test system: The db/db mice are animals that are said to display symptoms of human diabetic nephropathy and there are complete background data on them.

### Environmental Conditions of a Breeding Room

Breeding room: Breeding room S-202

Temperature: from 20 to 26° C. (actually measured temperature: from 21.5 to 24.5° C.)

Humidity: from 35 to 75% (actually measured humidity: from 44.5 to 71.5%)

Ventilation frequency: from 15 to 25 times/hour

Illumination time: 12 hours/day (from 7:00 to 19:00)

### <Test Item and Method>

Selection and grouping method of animals supplied for the test

Prior to potential application, 27 male mice which had showed a steady weight increase and normal performance status during the medical inspection and habituation term were selected and grouped by a method in accordance with the stratified random sampling method based on weight. The surplus animals were eliminated from the test system.

### Load of Potential (Exposure)

A potential generated by a potential therapy apparatus is applied to each mouse for 2.5 hours once a day during the application term (12 weeks in a row). Three mice are simultaneously housed in the potential therapy apparatus and load of potential is performed to each group (consisting of 9 mice) in three cycles while staggering the loading time. Mice in the control group are each similarly housed in the potential therapy apparatus for 2.5 hours once a day, but no potential was loaded thereto (non load group).

### Evaluation Method

The effects brought about by load of potential were evaluated based on weight, blood sugar level, urinary albumin and 8-hydroxydeoxyguanosine (8-OHdG) levels, hematological test, hematochemical test, histopathological test and histopathological test of kidney.

### Measurement of Body Weight

Frequency of measurement: Before load of potential and every week after load of potential is started.

Measuring method: Each mouse was weighed using an electronic even balance.

### Histopathological Test

Testing period: After the loading term

Testing method: After completion of collection of whole blood, the mouse was dissected in accordance with the pathological method and the intracranial, intrapleural and interperitoneal organs were macroscopically observed.

### Evaluation Results

Weight: (refer to Table 1. Table 1 is shown as a graph in FIG. 13)

It is known that db/db mice, that is, diabetic nephropathy model mice, develop obesity due to overeating when they are only eight to nine week old. It has been confirmed that the weight (from 40.2 to 45.7 g) of nine-week-old mice (at the time of grouping) of the non load group, alternating current group and superpose (−) group became considerably higher (more obese) than the reported weight (from 25 to 28 g) of normal (db/+m) mice of the same age.

The weight of the non load group using the db/db mice showed an increase from Week 1 (Pre) to Week 8 (52.2±1.0 g), showed a slight decrease until Week 13, and then restored balance. Blood was collected under anesthesia with ether on Week 4, Week 8 and Week 12 to measure their blood sugar level so that a weight reduction was observed one week after each blood collection.

There was not significant difference in the weight, in each week, of the nine-week-old db/db mice subjected to the load of potential for 12 weeks in a row between the alternating current group and non load group. This has suggested that the load of an alternating current potential has no influence on the weight change of the mice.

On the other hand, a significant reduction in the weight of the mice in the superpose (−) group occurred in Week 5 and from Week 7 to Week 12 compared with the non load group. This has suggested that the application of the superposed (−) potential to the db/db mice has an effect of suppressing their weight increase or reducing their increased weight, thus controlling the obesity of the db/db mice.

### Histopathological Test

Table 2 shows the results of the histopathological test. In Table 2, +++ means severe obesity. It has been found from this table that the db/db mice, that is, diabetic nephropathy model mice are observed to have obesity when they are only eight weeks old and some ten-week-old mice get a cataract. All of the nine cases in the non load group are recognized to get fat deposition (obesity) and cataract according to the results of autopsy. Also in all the cases of the alternating current group and superpose (−) group subjected to the load of potential for 12 weeks in a row (for 2.5 hours once a day) from when they were nine weeks old to when they were 21 weeks old, fat deposition (obesity) and cataract were observed. Suppression of fat deposition was however observed as a result of the load of superposed (−) potential, suggesting that it has an effect of improving obesity.

Judging from the fact that the load of potential by using the potential application apparatus did not cause any change in the organs and tissues (Table 3), it is presumed that the load of an alternating current potential or that of a superposed (−) potential has no influence on the organs and tissues.

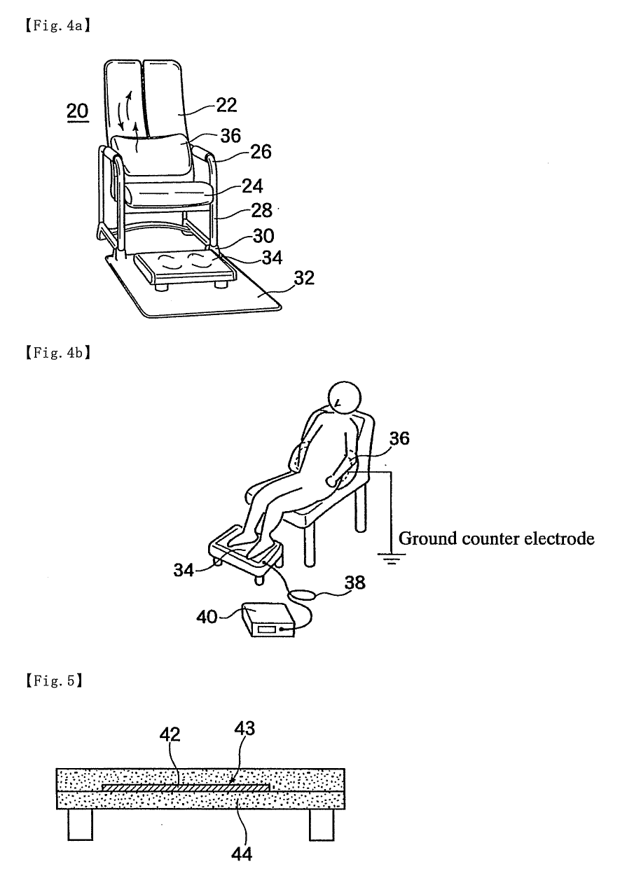

FIG. 4(a) is a schematic view of a chair-type potential application apparatus. In this drawing, a chair is indicated by the reference numeral 20. The chair 20 is equipped with a back portion 22, a seat portion 24, an arm portion 26 and a leg portion 28. An insulation shoe 30 is attached to the foot portion of the leg portion and an insulation mat 32 is attached to the insulation shoe. The insulation mat is laid on a floor. A stool which will serve as a conducting electrode plate 34 is placed on the insulation mat. A ground electrode 36 which will serve as a counter electrode of the conducting electrode plate is attached to the back portion so that it will be slidably moved up and down. The conducting electrode plate is connected to a controller 40 via a conducting code 38. A superposed waveform potential is applied to the conducting electrode plate. FIG. 4(b) is a schematic view illustrating a person sitting on the chair while placing his or her feet on the conducting electrode plate and having the ground electrode attached to the lumbar or knee. The ground electrode 36 is preferably installed, for example, at a site where many brown adipose tissues are present.

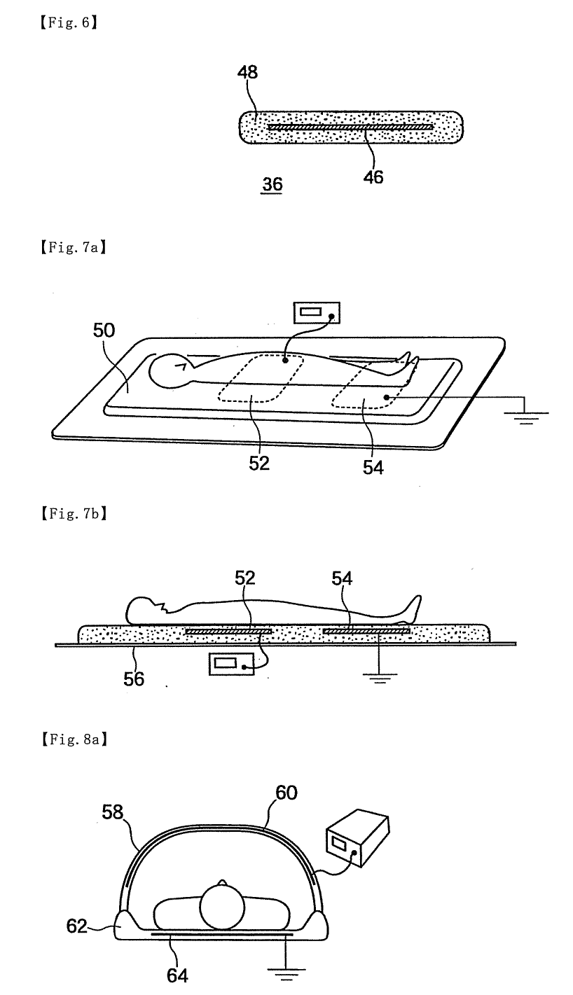

FIG. 5 is a cross-sectional view of the structure of a conducting electrode 34 obtained by covering a plate 42 made of metals, carbon or the like with an insulator 44. FIG. 6 is a cross-sectional view of the structure of a ground electrode 36 obtained by covering a conductor plate 46 with an insulator 48.

FIG. 7(a) is a schematic view of a mat-type potential application apparatus. In this drawing, a mat laid on a futon or the like is indicated by the reference numeral 50. The mat is equipped with a conducting electrode portion 52 and a ground electrode portion 54 serving as a counter electrode of the conducting electrode portion. FIG. 7(b) is a cross-sectional view of the potential application apparatus of FIG. 7(a). The mat 50 is laid over an insulator member 56 such as insulating vinyl, wood plate or the like. The mat 50 is made of urethane. The conducting electrode 52 and ground electrode 54 are placed on the lumbar portion and leg portion of a person lying on the mat, respectively.

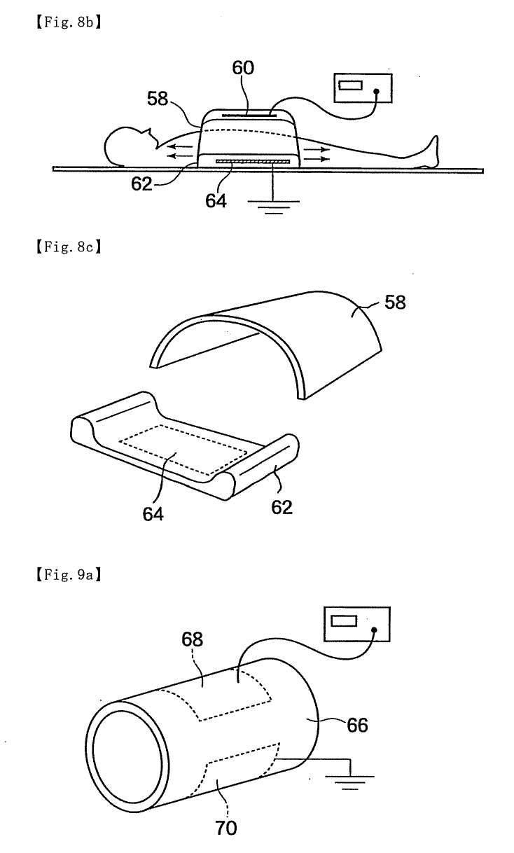

FIG. 8(a) is a schematic view of a dome-type potential application apparatus. This drawing illustrates a conducting electrode 60 attached to a dome-type roof member 58, a bed 62 over which a person connected to this electrode is to be laid, and a ground electrode 64 provided in this bed. FIG. 8(b) is a schematic view illustrating the apparatus disposed at the leg portion of a person lying on the bed. FIG. 8(c) is a partial exploded view of the apparatus.

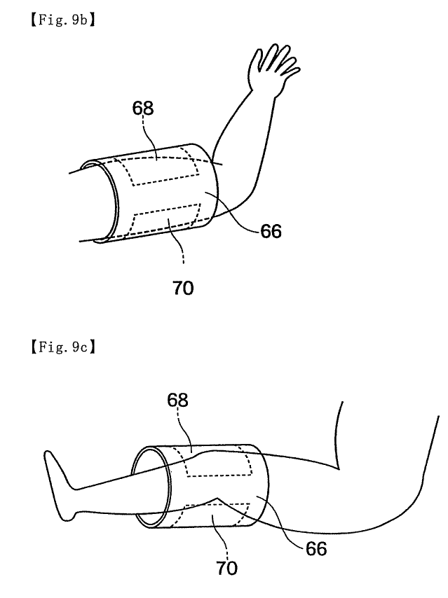

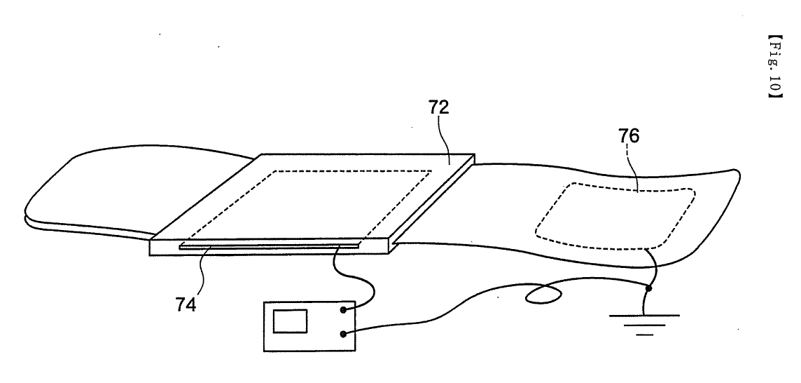

FIG. 9(a) is a schematic view of a hollow-cylinder-type potential application apparatus. In the apparatus shown in this drawing, a hollow cylindrical member 66 is provided with a conducting electrode 68 and a ground electrode 70. FIG. 9(b) and FIG. 9(c) are schematic views illustrating the apparatus attached to the arm and leg portions, respectively.

FIG. 10 is a schematic view of a roll-type potential application apparatus. In the drawing, a roll member 72 has, at the center and end thereof, a conducting electrode 74 and a ground electrode 76, respectively. This apparatus is attached to, for example, an abdominal portion or leg portion.

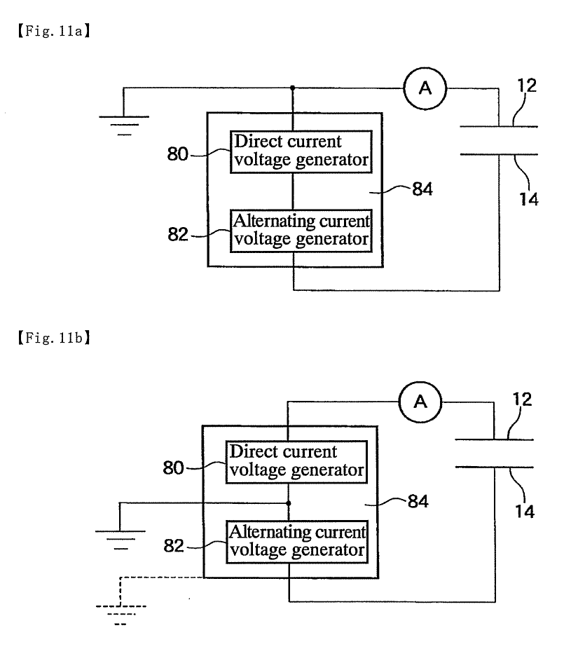

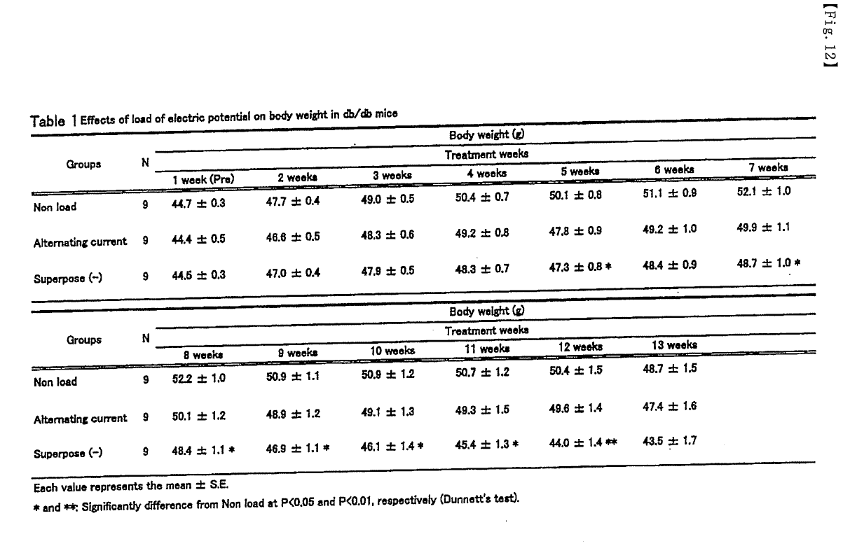

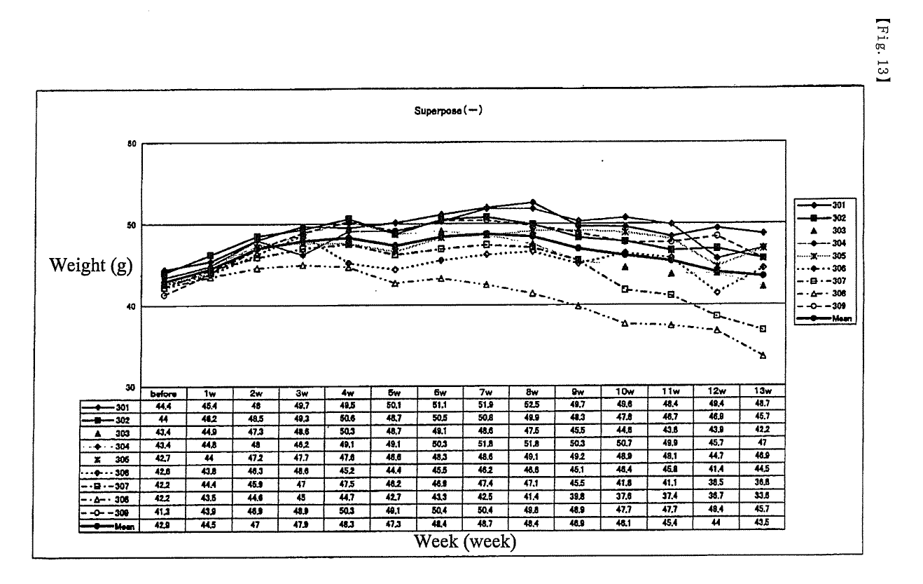

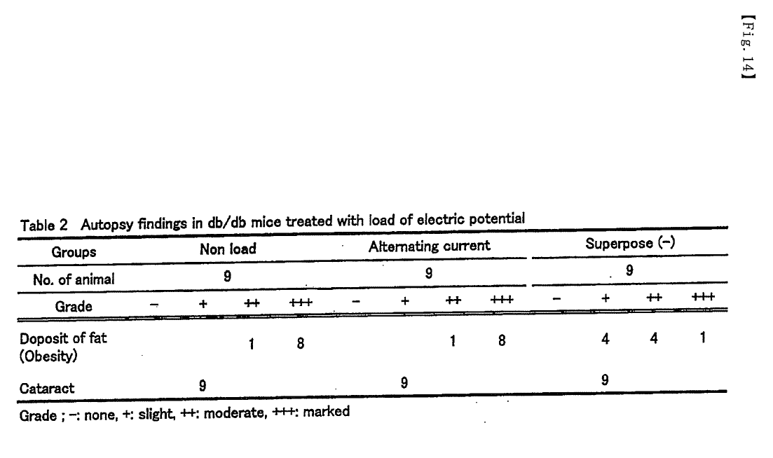

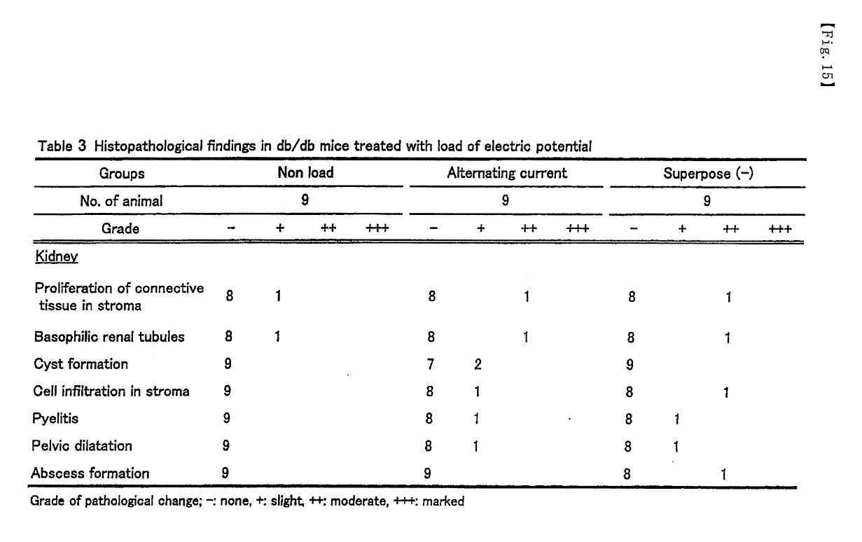

Circuits for forming a superposed waveform potential to be applied to a drive electrode are illustrated in FIGS. 11(a) and 11(b).

In FIG. 11(a), a controller of a potential application apparatus is equipped with a direct current voltage generator and an alternating current voltage generator. The cell deposition inhibiting apparatus has a first electrode and a second electrode disposed vertically opposite to each other. In the potential application apparatus 10 in the drawing, the first electrode 12 is used as a ground electrode and the second electrode 14 is used as a drive electrode. A superposed waveform potential of the above-described direct current and alternating current is applied to the drive electrode 14.

In FIG. 11(b), a negative direct current potential of from 100 to 500 V, preferably from 200 to 400 V and an alternating current potential of from 50 to 1500 V may be applied to the first electrode 12 and second electrode 14, respectively. This drawing illustrates a circuit for applying an alternating current potential and a negative direct current potential to the first electrode and the second electrode, respectively, thereby generating a superposed waveform potential. In this drawing, a ground potential is located between the second electrode or direct current voltage generator and the alternating current voltage generator.

### INDUSTRIAL APPLICABILITY

The potential application apparatus according to the present invention can be used for obesity preventive health appliances for preventing obesity which will cause various adult diseases or chronic diseases, appliances for cosmetic purposes, and obesity preventive appliances to be used in hospitals or clinics.

1. A potential application apparatus for applying a superposed waveform potential consisting of a negative direct current potential plus an alternating current potential to a human body at a predetermined frequency while insulating the human body.

2. A potential application apparatus for applying a superposed waveform potential consisting of a negative direct current potential plus an alternating current potential of from 50 to 1500 V to a human body at a frequency of from 5 to 100 kHz, preferably from 30 to 40 kHz, while insulating the human body.

3. A potential application apparatus for applying a superposed waveform potential, which has been consisted of a negative direct current potential plus an alternating current potential of from 50 to 1500 V and controlling a ratio of a potential on the positive side to a potential on the negative side to 1:2 or less, to a human body at a frequency of from 5 to 100 kHz, preferably from 30 to 40 kHz, while insulating the human body.

4. A potential application apparatus, comprising a first electrode for applying a superposed waveform potential, which has been consisted of a negative direct current potential plus an alternating current potential of from 50 to 1500 V, at a frequency from 5 to 100 kHz, preferably from 30 to 40 kHz, and a second electrode on the side opposite to the first electrode, which serves as a ground electrode.

5. A potential application apparatus, which comprises a first electrode for applying an alternating current potential of from 50 to 1500 V at a frequency of from 5 to 100 kHz, preferably from 30 to 40 kHz and a second electrode on the side opposite to the first electrode for applying a negative direct current potential; and forms a superposed waveform potential.

6. A potential application apparatus according to any one of claims 1 to 5, wherein a composite waveform potential obtained by adding a fluctuation waveform to the superposed waveform potential is applied.

7. A potential application apparatus according to any one of claims 1 to 5, wherein the electrode for applying the superposed waveform potential is attached to an adipose cell deposition inhibited site.

8. A potential application apparatus according to claim 7, wherein a band roll to which the electrodes have been attached is wound around the adipose cell deposition inhibited site.
